(19) United States
(12) Patent Application Publication
(10) Pub. No.: US 2017/0238829 A1
(43) Pub. Date: Aug. 24, 2017

(54) CATHETER GRIP WITH HINGE

(71) Applicant: Steven Parker, Spokane, WA (US)

(72) Inventor: Steven Parker, Spokane, WA (US)

(21) Appl. No.: 15/236,405

(22) Filed: Aug. 13, 2016

Related U.S. Application Data
(63) Continuation-in-part of application No. 15/051,513, filed on Feb. 23, 2016.

(51) Int. Cl. A61B 5/042; A61M 25/01; A61B 5/00

(52) U.S. Cl. CPC A61B 5/042; A61B 5/6852; A61M 25/013

(57) ABSTRACT

The present disclosure provides a catheter grip device that secures to a catheter shaft and provides easier manipulation of the catheter shaft for the user. For example, the catheter shaft, such as for an Electrophysiology catheter, may insert into a patient's blood vessel. From here, the user, such as the physician, may need to manipulate, re-position, rotate, or otherwise maneuver a distal end of the catheter shaft while inserted inside the patient's blood vessel. In this regard, by the catheter grip being securely attached to the catheter shaft, the user is able to manipulate, rotate, etc. the catheter shaft by rotating and manipulating the catheter grip. The catheter grip reduces strain on the various muscles of the user's hand, and also provides more accurate manipulation of the catheter shaft.

### CROSS-REFERENCE TO RELATED APPLICATIONS

This application is a Continuation-In-Part Application of application Ser. No. 15/051,513, filed Feb. 23, 2016, the entire disclosure of which is hereby incorporated herein by reference.

### BACKGROUND

Patients undergo Electrophysiology tests in order to identify the cause of an arrhythmia. During an electrophysiology test, a catheter shaft is inserted into the patients blood vessel, such as via the groin, in which a doctor sends electrical pulses through the catheter shaft that cause the heart to beat at various speeds. The heartbeat rhythms are picked-up and recorded by a catheter device (e.g., cardiac mapping), and the doctor is able to identify the cause of the arrhythmia, which may then be destroyed via a catheter ablation.

The catheter shaft is inserted and controlled by a proximal handle attached to the catheter shaft. An electrical cable is inserted into the proximal end of the handle to control the electrical pulses sent through the catheter. In order to manipulate a distal end of the catheter shaft, the doctor may manipulate the catheter handle and/or grasp the catheter shaft itself and rotate it, such as by using an index finger and thumb. By rotating as such, strain and muscle soreness or tightness may result because the doctor has to continue rotating the catheter shaft using only the two fingers throughout the procedure. Strain on doctor's muscle may affect the doctor's ability to adequately control the catheter shaft.

### SUMMARY

A catheter grip device that provides easier control and thereby less strain on the doctor's muscles is disclosed herein. The catheter grip is secured to the electrophysiology catheter shaft with pressure from a receiving portion attached to a threaded cap, wherein the threaded cap rotates about the threads of the receiving portion, thereby securing the catheter shaft in place. Once the catheter shaft is secured to the catheter grip, the doctor is able to grasp the catheter grip in order to rotate the catheter shaft, as opposed to only being able to grip the catheter shaft itself. In this regard, the doctor can rotate the catheter grip when he or she needs to manipulate the distal end of the catheter shaft. The doctor can move the distal end of the catheter shaft forward and backward relative to the patient and/or rotate the distal end of the catheter shaft within the blood vessel of the patient. The ability to manipulate the catheter shaft using the catheter grip may also allow the muscles in the wrist, forearm, and elbow to absorb the strain that would typically be solely on the muscles within the hand of the doctor, such as the opponens pollicis and adductor pollicis.

A catheter grip device is disclosed herein, the catheter grip includes a first arm, the first arm including a first surface and a first recess; a second arm, the second arm including a second surface and a second recess; wherein the first arm and the second arm are at least partially connected to each other to form a connection, and the connection causes the catheter grip to be in an opened position and a closed position, such that in the opened position the first and second surfaces do not contact each other, and in the closed position the first and second surfaces at least partially contact each other. As a further embodiment, the first and second arms are connected to each other at a hinge, and the hinge provides the catheter grip to operate in the opened and closed position.

As another example, in the closed position the first recess and the second recess are positioned adjacent to each other, such that a bore is created in the catheter grip. As a further example, a catheter shaft is positioned between the first recess and the second recess when the catheter grip is in the closed position. As another example, a tab positioned on the first arm; and a notch positioned on the second arm, wherein in the closed position the tab connects with the notch to securely lock the catheter grip in place. In that example, the tab extends from the first arm such that a space is defined between the tab and the first arm, and the tab pivots at a point of connection of the tab and the first arm to provide a release mechanism of the tab from the notch. As an even further example, a size of the bore is adapted to a particular sized catheter shaft, such that only catheter shafts with a required diameter is able to fit within the bore when the catheter grip is in the closed position. As a further embodiment, the first and second arms include ridges to provide grip for the catheter grip, wherein the ridges are positioned on an outside surface of the first and second arms.

As another example, a kit is disclosed herein, the kit includes a first catheter grip and a second catheter grip that is separate from the first catheter grip, wherein both of the first and second catheter grips comprise: a first arm, the first arm including a first surface and a first recess; a second arm, the second arm including a second surface and a second recess; wherein the first arm and the second arm are at least partially connected to each other to form a connection, and the connection causes the catheter grip to be in an opened position and a closed position, such that in the opened position the first and second surfaces do not contact each other, and in the closed position the first and second surfaces at least partially contact each other, wherein in the closed position the first recess and the second recess are positioned adjacent to each other, such that a bore is created in the catheter grip.

As a further example, a size of the bore of the first catheter grip is different than a size of the bore of the second catheter grip. As another example, the first and second arms are connected to each other at a hinge, and the hinge provides the catheter grip to operate in the opened and closed position.

### BRIEF DESCRIPTION OF THE DRAWINGS

FIG. 1A-C illustrate various views of a catheter grip in accordance with aspects of the disclosure.

FIG. 2 depicts a placement of a catheter shaft within the opened catheter grip in accordance with aspects of the disclosure.

FIG. 3 depicts the catheter grip in a closed position and the catheter shaft positioned within a bore of the catheter grip in accordance with aspects of the present disclosure.

FIGS. 4A-B illustrate another embodiment of the catheter grip with a living hinge in accordance with aspects of the present disclosure.

FIGS. 5A-D illustrate another embodiment of the catheter grip in accordance with aspects of the present disclosure.

### DETAILED DESCRIPTION

The aspects, features and advantages of the present disclosure will be appreciated when considered with reference to the following description of preferred embodiments and accompanying figures. The following description does not limit the disclosure; rather, the scope is defined by the appended claims and equivalents. While certain processes in accordance with example embodiments are shown in the figures as occurring in a linear fashion, this is not a requirement unless expressly stated herein. Different processes may be performed in a different order or concurrently.

The present disclosure describes a catheter grip device that securely attaches to a portion of a catheter shaft as an add-on component, and provides easier gripping and manipulation of the catheter shaft by an individual, such as a doctor. The catheter grip includes a first arm and a second arm that connects to each other at a hinge, the hinge allowing the first and second arms to open and close. In an opened position the first and second arms swing about the hinge, which thereby provides an opening or otherwise access to an inside of the catheter grip. A catheter shaft may be placed between the first and second arms at this point. In a closed position a plane of the first arm and a plane of the second arm come into contact with each other, thereby closing the opening and access to the inside of the catheter grip. When closed, the catheter shaft is positioned between two recesses, a first recess on the first arm and a second recess on the second arm. When the catheter grip is in the closed position, the two recesses when together form a bore, and the perimeter thereof encapsulates the catheter shaft and thereby holds the catheter shaft in place. In addition, the first and second arms may include a tab and notch type locking mechanism that securely keeps the catheter grip in the closed position. The tab and notch mechanism may be positioned opposite the hinge or where the first and second arms come together.

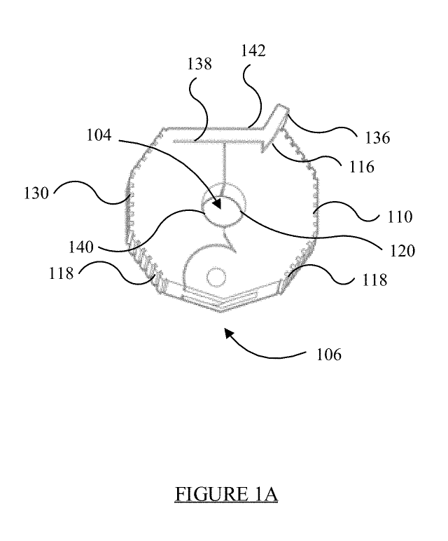

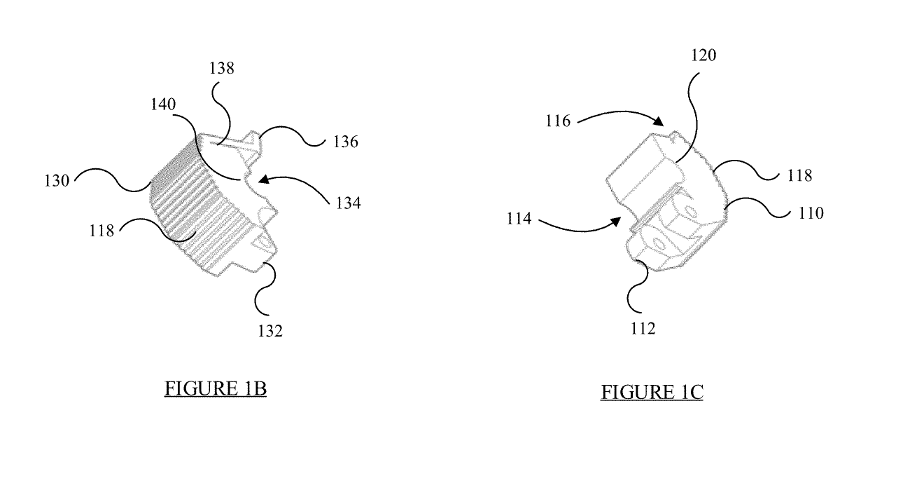

FIG. 1A is a perspective view of catheter grip 102. As illustrated in FIG. 1A, catheter grip 102 includes first arm 130 and second arm 110, which are connected to each other via hinge 106. First and second arms 130 and 110 may be comprised of plastic, metal, or any material that provides sufficient structure so catheter grip 102 is able to function as described herein.

As depicted in FIGS. 1B-C, first arm 130 includes first leaf 132 and second arm 110 includes second leaf 112, which come together to form hinge 106. In this regard, a pin (not shown) may extend through respective bores on first leaf 132 and second leaf 112, to form the pivoting section of hinge 106. Alternatively, a protrusion may extend from one or two sides of first leaf 132, which enter corresponding recesses in second leaf 112. Although a hinge is depicted herein, it should be understood that other connecting mechanisms may be implemented as well, such that the two arms are at least partially connected to each other, such as on or substantially near the ends thereof. For example, any method of connecting first arm 130 to second arm 110, and allows the two arms to move about in an opened and closed position, may operate with the present disclosure, including joints, the two arms being integrally connected with each other, a tab and notch, friction fit, etc.

FIG. 1B illustrates a first recess 134 on a surface of first arm 130, the first recess of which is shaped by perimeter 140. Similarly and as illustrated in FIG. 1C, second recess 114 is positioned on a surface of second arm 110, the second recess of which is shaped by second perimeter 120. When in the closed position, first and second perimeters 140 and 120 form bore 104, which forms a substantial circular shape. As discussed in further detail below, a catheter shaft may be positioned in between first and second recesses 134 and 114, and held in place by first and second perimeters 140 and 120 when catheter grip 102 is in the closed position. It should be understood that the perimeters and the formed bore is not limited to a circular shape, but may be any shape that properly secures any shaft in place.

Catheter grip 102 also implements a locking mechanism to securely lock catheter grip 102 in the closed position. For example, catheter grip 102 includes a tab and notch mechanism where tab 142 locks into notch 116. First arm 130 includes tab 142 that extends from a portion thereof. A space 138 is between tab 142 that allows tab 142 to be pulled about a pivot, which thereby unhooks or disengages tab 142 from notch 116. Thus, pulling back tab 142 acts as a release mechanism. The pivoting section of tab 142 is the point of connection between the body of first arm 130 and tab 142. Tab 142 includes lip 136 that corresponds to a shape of notch 116, which is located on first arm 110. Tab 142 locks into place by lip 136 of tab 142 securely mating with notch 116.

After the user releases or otherwise lets go of tab 142, it reverts back to its original position based on tension, so that tab 142 can again mate with notch 116 in the future. It should be understood that other locking mechanisms may also be employed, such as friction fit, press-fit, etc.

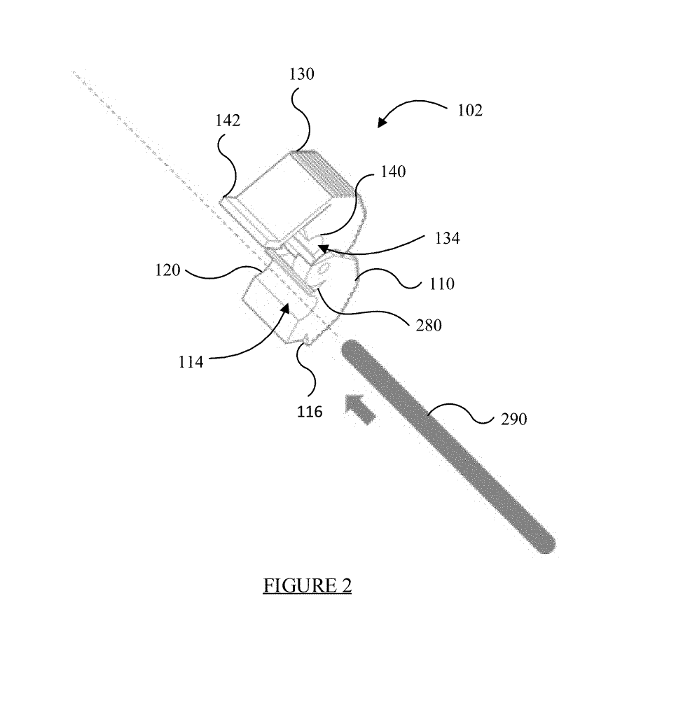

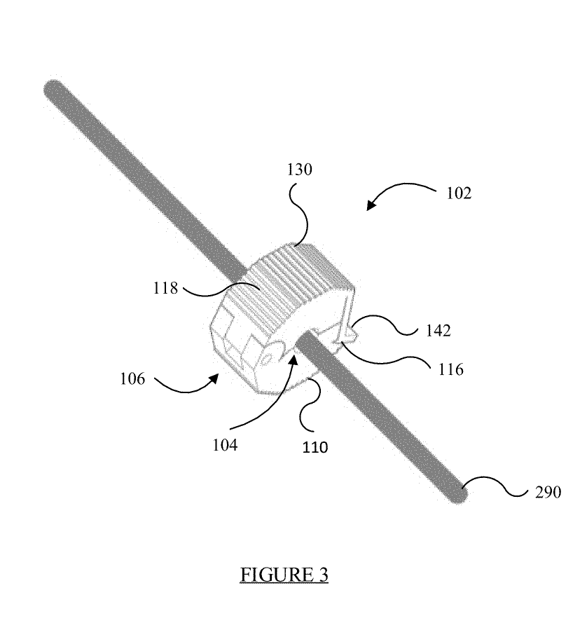

As illustrated in FIG. 2, catheter shaft 290 is inserted between first and second arms 130 and 110. As one example, the user may insert shaft 290 such that it is positioned within one of first or second recesses 134 or 114, thereby in contact with first or second perimeters 140 or 120. Furthermore and as illustrated in FIG. 2, a ridge 280 is placed near the hinge and in between first and second arms 130 and 110, to provide a stop to locate the catheter shaft into the recess. For example, the user may simply place catheter shaft 290 in front of ridge 280, which helps hold catheter shaft 290 in place while the user closes catheter grip 102. As another example, the user may simply position shaft 290 between first and second arm 130 and 110 and then close catheter grip 102 about hinge 106, such that catheter shaft 290 is ultimately positioned within bore 104.

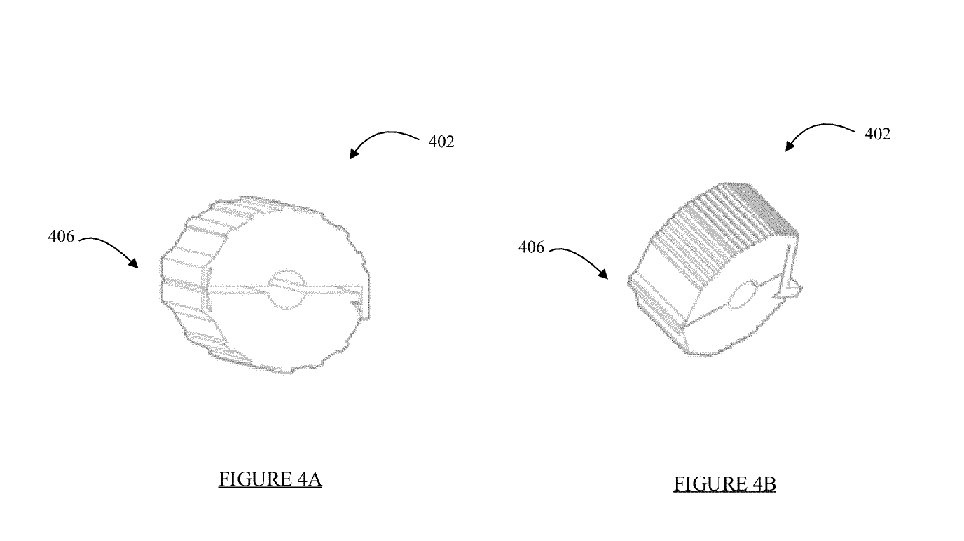

As shown in FIG. 3, catheter shaft 290 is securely positioned within bore 104 and in either complete contact or substantial contact with first and second perimeters 140 and 120. Shaft 290 is locked in place via the locking mechanism employed, which in this scenario is lip 136 of tab 142 mating with notch 116.

Catheter grip 102 covers only a small portion of catheter shaft 350, thus not being obstructive or a hindrance to the user. Catheter grip 102 may be positioned at any point on catheter shaft 290, so long as there is sufficient length of catheter shaft 290 to be inserted within a blood vessel of a patient. The user is able to grasp catheter grip 102 with their hand and fingers, thereby providing him or her ease to manipulate, rotate, insert, remove, etc. catheter shaft 290 when inserted within the patient.

Without catheter grip 102 the user may be restricted to grasping catheter shaft 290 itself when manipulating or repositioning catheter shaft 290 within the patient. For example, typically the user may use his or her thumb and index finger to apply pressure to catheter shaft 290 in order to manipulate a distal end of catheter shaft 290. By only gripping catheter shaft 290 this may cause additional strain to the user's hand muscles, such as the Adductor Pollicis and Opponens Pollicis muscles. Catheter grip 102 helps remove strain from the various muscles in the hand of the user by allowing the user to grasp a more robust device, catheter grip 102, which is not only easy to manipulate but may also allow the user to be more accurate and careful when manipulating catheter shaft 290 within the patient. In this regard, catheter grip 102 is not only useful in terms of reducing strain on the muscles of the user, but also increases safety for the patient.

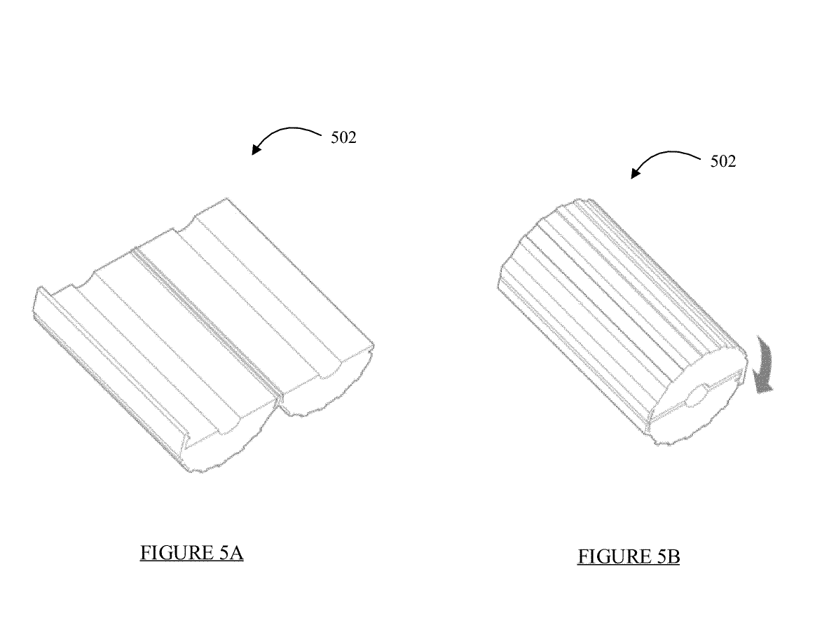

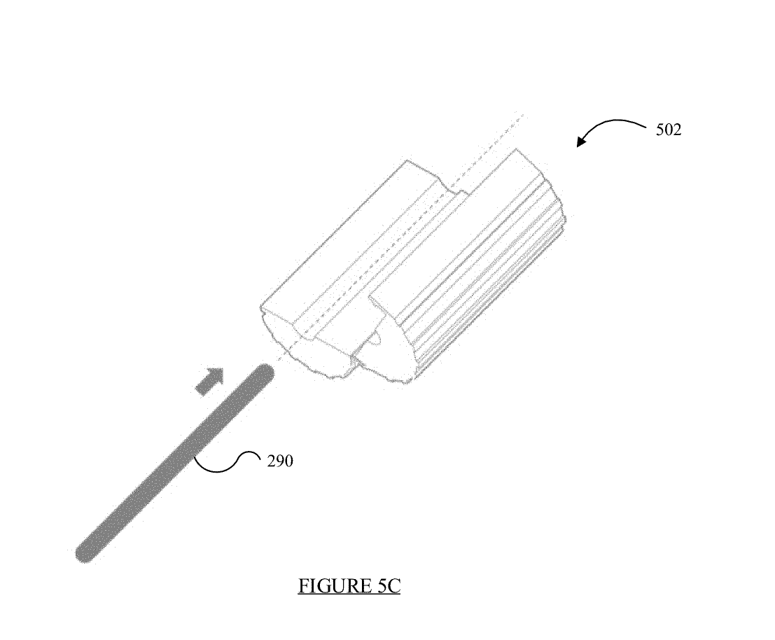

As a further embodiment, FIGS. 4A-B illustrate catheter grip 402, which is configured and operates similarly to catheter grip 102, but instead implements a living hinge 406. As illustrated in FIGS. 4A-B, living hinge 406 may allow for the entire catheter grip 402 apparatus to be manufactured at once, given that living hinge 406 may have been manufactured as one piece with both arms of the catheter grip. Living hinge 406 may also include a stop that prohibits the hinge from excessively bending in the opened position.

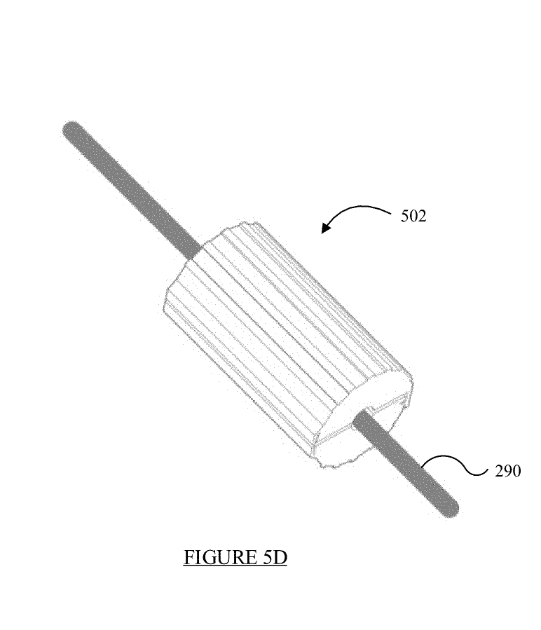

As illustrated in FIGS. 5A-D, other shapes and designs of the catheter grip is also possible. For instance, catheter grip 502 depicted in FIGS. 5A-D is designed similarly and includes similar functionality as catheter grip 102 described above, but the catheter grip 502 is longer in the longitudinal direction. The longer length of catheter grip 502 may provide additional surface area for the user to grip when using the device.

As a further embodiment, there may be multiple catheter grips similar to the catheter grip that have varying bore sizes. For example, the diameter of catheter shafts or any type of shafts that the catheter grip is used for may vary in sizes. Therefore, various types of catheter grips with varying bore sizes may be manufactured to accommodate a particular sized diameter of a shaft. As a further example, multiple catheter grips with varying sized bores may be packaged together such that a user or company can purchase one complete kit or package with all necessary catheter grip sizes.

As another embodiment, a single catheter grip may be configured to accommodate various sized catheter shafts. For example, a spongy insert may be positioned within and around first diameter 140 and second diameter 120, such that the spongy material is able to hold thinner diameters of shafts in place, and then easily contract when a thicker sized diameter of a shaft is used. The spongy insert may be comprised of various types of material, including silicone, rubber, etc. It should be understood that the insert should not be limited to a spongy type material, but rather any material that is capable of contracting and/or resistant enough to accommodate the shape of the catheter shaft, but has enough substance to hold the catheter shaft in place. Other methods may also be implemented that allow for a single catheter grip to accommodate multiple different sizes of catheter shafts.

As a further embodiment, the catheter grips described herein may include a rubber insert within one or both of the first or second perimeters, which thereby provides additional friction and secures the catheter shaft in place.

Advantages of the present disclosure include reducing the amount of strain on the user's hand muscles when operating and manipulating the catheter shaft. For instance, instead of the user having to grasp and manipulate the catheter shaft between his or her fingers, such as the thumb and index finger, the user is able to fully grasp the catheter grip and any manipulation of the catheter grip translates directly to the catheter shaft itself. In this regard, instead of all of the strain being on muscles within the hand, by being able to grasp the catheter grip the muscles in the wrist, forearm, and elbow may be utilized, thereby absorbing the strain on the muscles within the hand of the user. Furthermore, the present disclosure may also provide additional accuracy and overall control when manipulating the catheter shaft, thereby also increasing safety to the patient.

Most of the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A catheter grip, comprising:
a first arm, the first arm including a first surface, a first recess, and a tab; and
a second arm, the second arm including a second surface, a second recess, and a notch,
wherein the first arm and the second arm are at least partially connected to each other to form a connection, and the connection causes the catheter grip to be in an opened position and a closed position, such that in the opened position the first and second surfaces do not contact each other, and in the closed position the first and second surfaces at least partially contact each other; and
a ridge positioned on the second surface of the second side arm, wherein the ridge is an indent within the second surface, the indent configured to provide a stop for a catheter when the catheter grip is in the opened position.

2. The catheter grip of claim 1, wherein in the closed position the first recess and the second recess are positioned adjacent to each other, such that a bore is created in the catheter grip.

3. The catheter grip of claim 1, wherein a catheter shaft is positioned between the first recess and the second recess when the catheter grip is in the closed position.

4. The catheter grip of claim 1,
wherein in the closed position the tab connects with the notch to securely lock the catheter grip in place.

5. The catheter grip of claim 4, wherein the tab extends from the first arm such that a space is defined between the tab and the first arm, and the tab pivots at a point of connection of the tab and the first arm to provide a release mechanism of the tab from the notch.

6. The catheter grip of claim 2, a size of the bore is adapted to a particular sized catheter shaft, such that only catheter shafts with a required diameter is able to fit within the bore when the catheter grip is in the closed position.

7. The catheter grip of claim 1, wherein the first and second arms include ridges to provide grip for the catheter grip, wherein the ridges are positioned on an entire outside radial surface of the first and second arms except for the tab, notch, and hinge components.

8. The catheter grip of claim 1, wherein the first and second arms are connected to each other at a hinge, and the hinge provides the catheter grip to operate in the opened and closed position.

9. A kit having components contained within the same package, the kit comprising:
a first catheter grip and a second catheter grip that is separate from the first catheter grip, wherein both of the first and second catheter grips comprise:
a first arm, the first arm including a first surface, a first recess, and a tab; and
a second arm, the second arm including a second surface, a second recess, and a notch,
wherein the first arm and the second arm are at least partially connected to each other to form a connection, and the connection causes the catheter grip to be in an opened position and a closed position, such that in the opened position the first and second surfaces do not contact each other, and in the closed position the first and second surfaces at least partially contact each other, wherein in the closed position the first recess and the second recess are positioned adjacent to each other, such that a bore is created in the catheter grip; and
a ridge positioned on the second surface of the second side arm, wherein the ridge is an indent within the second surface, the indent configured to provide a stop for a catheter when the catheter grip is in the opened position.

10. The kit of claim 9, wherein a size of the bore of the first catheter grip is different than a size of the bore of the second catheter grip.

11. The kit of claim 9, wherein the first and second arms are connected to each other at a hinge, and the hinge provides the catheter grip to operate in the opened and closed position.
